(19) United States
(12) Patent Application Publication
(10) Pub. No.: US 2007/0025944 A1
(43) Pub. Date: Feb. 1, 2007

(54) Cosmetic compositions useful for lengthening lashes

(75) Inventors: Sue Feng, Edison, NJ (US); Stacy Sterphone, Hillsborough, NJ (US); Kavita Patel, North Brunswick, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL, Paris (FR)

(21) Appl. No.: 11/194,841

(22) Filed: Aug. 1, 2005

(51) Int. Cl. A61K 8/73

(52) U.S. Cl. 424/70.13; 424/63

(57) ABSTRACT

Disclosed are cosmetic compositions containing xanthan gum and a non-gelling polysaccharide, and methods of making and using same.

### BACKGROUND OF THE INVENTION

The cosmetic industry focuses much of its efforts, in regards to mascara, on increasing two fundamental properties, namely enhancing volume or thickness of eyelashes and extending length of wear.

U.S. Pat. No. 5,874,072 teaches mascara containing a mixture of water-insoluble copolymers in the form of an aqueous emulsion with water-soluble film-forming polymers. U.S. Pat. No. 6,248,336 teaches mascara compositions with improved wear characteristics in the form of an emulsion comprising an insoluble polymeric material in an aqueous emulsion, and lipophilic oil components including a polyvinylpyrrolidone hexadecane copolymer.

There have also been advancements in creating an effect of eyelashes with increased volume. For example, U.S. Pat. No. 6,726,917 discloses a mascara containing fibers, pigments, and at least two film formers: at least one tacky film former soluble or dispersible in water and at least one tacky film former soluble in oil. Similarly, U.S. Pat. No. 6,503,521 teaches mascara that enhances volume via the use of three film formers, namely: at least one tacky film former soluble or dispersible in water; at least one tacky film former soluble in oil; and at least one additional water-soluble or water-dispersible film former.

### SUMMARY OF THE INVENTION

A first aspect of the present invention is directed to a mascara composition comprising an aqueous phase, a liquid fatty phase, a structuring agent such as a wax, a film-forming agent, xanthan gum and a non-gelling polysaccharide.

A second aspect of the present invention is directed to a method of extending wear and/or enhancing volume of eyelashes, comprising applying to eye lashes a mascara composition comprising xanthan gum and a non-gelling polysaccharide.

A third aspect of the present invention is directed to a method for applying make-up to eye lids or lips comprising applying to eye lids or lips a cosmetic composition comprising xanthan gum and a non-gelling polysaccharide.

### DETAILED DESCRIPTION

Xanthan is a high molecular weight polysaccharide or hydrophilic colloid (also known as a gum) that is produced by fermentation of various species of the Xanthomonas, such as X. carotae, X. campestris, X. incanae, X. begoniae and X. malvacearum. To alternate glucose residues is attached a trisaccharide side chain that contains a first mannose, a glucuronic acid and a second mannose residue. Xanthan is a glucomannan—the first mannose residue is acetylated, while approximately half of the terminal mannose residues of the side chains contain a pyruvate substituent. Xanthan is also known to be acid stable and heat resistant. It can be used in compositions having a pH ranging from about 2 to about 10. The viscosity of xanthan generally ranges from about 1,000-2,000 cP (measured as a 1% solution at 25° C. using a Brookfield viscometer).

The non-gelling polysaccharides useful in the present invention include glucomannans and galactomannans alike. An example of a non-gelling glucomannan is konjac mannan gum, which is a linear polysaccharide derived from the konjac tuber of the amorphophallus species found in East Asia. Galactomannans are a class of polysaccharides composed of galactose and mannose, generally referred to as gums. In general, the structure of the polymers consist of a linear chain of 1,4-linked βD-mannopyranosyl units with single-membered α-D-galactopyranosyl units joined by 1,6 linkages. Galactomannans vary in the extent and uniformity of substitution with respect to their galactose moieties. Galactomannans include but are not limited to guar and locust bean gums. In some embodiments, the galactomannan is locust bean gum (LBG), also known as carob or carob gum, which is a polysaccharide derived from the endosperm of the locust bean (Ceratonia siliqua). The backbone of LBG consists of β-1,4-linked mannose residues with single α-1,6-linked galactose side units, wherein the weight ratio of galactose to mannose is approximately 1:4. In guar gum, this ratio is approximately 1:2. See, U.S. Patent Pub. 2005/0164892. As taught in U.S. Patent Publication no. 2005/0158262, examples of galactomannans are disclosed in U.S. Pat. Nos. 3,589,578 and 4,031,307 (e.g., guar gums having trialkylammonium cationic groups, and guar gums modified with a salt, e.g., chloride of 2,3-epoxypropyltrimethylammonium).

Amounts of xanthan generally range from about 0.1% to about 5%, and preferably from about 0.1% to about 1.0% by weight of the composition. Amounts of the non-gelling polysaccharide, e.g., konjac mannan, guar or locust bean gum, generally range from about 0.1% to about 5%, and preferably from about 0.1 to about 1.0% by weight of the composition. The ratio of xanthan to the non-gelling polysaccharide generally ranges from about 1:10 to about 10:1. Xanthan and non-gelling polysaccharides are commercially available from numerous sources. For example, xanthan and locust bean gum can be obtained commercially as a pre-made mixture, from Nisshin Oil Mills, Ltd. (Japan) under the tradename Nomcort CG (a 50/50 mixture by weight). As taught in U.S. Patent Publication no. 2005/0158262, galactomannans also include those sold under the names Jaguar C13 S, Jaguar C 15, Jaguar C 17, and Jaguar C 162 by the company Rhodia.

Compositions of the present invention may also contain a moisturizer. Examples include sodium lactate; mannitol; amino acids; hyaluronic acid; lanolin; urea; petroleum jelly; and mixtures thereof. Other examples include polyols such as glycerin, diglycerin, triglycerin, polyglycerin, polyethylene glycol, ethylene glycol, diethylene glycol, triethylene glycol, propylene glycol, dipropylene glycol, hexylene glycol, 1,3-butylene glycol, 1,4-butylene glycol and sorbitol. A preferred moisturizer is glycerin. These agents are present in the compositions of the present invention in amounts generally ranging from about 1.0% to about 15%, and in some cases, from about 2.0% to about 10% by weight of the composition.

In some embodiments, the cosmetic compositions of the present invention are in the form of mascara. Applicants have discovered that mascara of the present invention, when applied to eyelashes, has extended wear and/or achieves enhanced or greater volume or thickness. Masacara may be formulated as washable or waterproof. The term washable mascara, as used herein, refers to compositions that may be removed with water and/or soap. These formulations are typically emulsions (e.g., of waxes in water) such as creams, or in some cases gels and cakes. Waterproof mascara, which require use of oils for removal, generally come in the form of dispersions of waxes in organic solvents, e.g., isododecane and petroleum distillate.

In other embodiments, the compositions of the present invention may be formulated as water-based lip compositions such as gloss, and compositions for application to the eye area such as eyeshadow.

The compositions of the invention contain a cosmetically acceptable carrier. The carrier contains a fatty phase and an aqueous phase. The fatty phase typically contains one or more of fatty substances chosen from oils, organic solvents, structuring agents such as waxes structuring polymers, pasty substances and mixtures thereof.

As disclosed herein, the term “liquid fatty phase” means refers to a non-aqueous medium that is liquid at room temperature (25° C.) and atmospheric pressure (760 mmHg, i.e., 105 Pa), comprising at least oil or oily liquid that are in general, are mutually compatible. Oily (oil soluble) liquids are included in the compositions of the present invention to provide desirable feel, spreadability, and/or other desirable characteristics. The inventive compositions may contain any cosmetically or dermatologically acceptable and, in general, physiologically acceptable oil, chosen in particular from carbon-based, hydrocarbon-based, fluoro and/or silicone oils, of mineral, animal, plant or synthetic origin, alone or as a mixture, provided that they form a homogeneous and stable mixture and provided that they are compatible with the intended use.

The term “hydrocarbon-based oil” means an oil mainly comprising carbon and hydrogen atoms and possibly at least one functional group chosen from hydroxyl, ester, ether and carboxylic functional groups. For example, the oils may have a viscosity ranging from 0.5 to 300,000 centipoise (cps), further for example, from 50 to 50,000 cps, and even further for example, from 100 to 100,000 cps. Examples of hydrocarbon-based oils include hydrocarbon-based oils of animal origin, such as perhydrosqualene; hydrocarbon-based plant oils such as liquid triglycerides of fatty acids of from 4 to 24 carbon atoms, for instance heptanoic or octanoic acid triglyceride, or alternatively sunflower oil, maize oil, soybean oil, marrow oil, grapeseed oil, sesame seed oil, hazelnut oil, apricot oil, macadamia oil, castor oil, avocado oil, caprylic/capric acid triglycerides, for instance those sold by the company Stearineries Dubois or those sold by the company Dynamit Nobel under the names Miglyol 810, 812 and 818, jojoba oil and shea butter; linear and branched hydrocarbons of mineral or synthetic origin, for example, liquid paraffin and derivatives thereof, petroleum jelly, polydecenes, polybutenes and hydrogenated polyisobutene, for example Parleam™; esters of lanolic acid, of oleic acid, of lauric acid or of stearic acid; fatty esters, such as isopropyl myristate, isopropyl palmitate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate or lactate, 2-octyldodecyl stearate, 2-octyldodecyl erucate, bis(2-ethylhexyl) succinate, diisostearyl malate, glyceryl triisostearate or diglyceryl triisostearate, isopropyl myristate, isostearyl isostearate and tridecyl trimellitate; higher fatty acids, such as myristic acid, palmitic acid, stearic acid, behenic acid, oleic acid, linoleic acid, linolenic acid or isostearic acid; higher fatty alcohols (e.g., having from 10-26 carbon atoms) such as ketanol, stearyl alcohol, oleyl alcohol, linoleyl alcohol, linolenyl alcohol, isostearyl alcohol or octyldodecanol; hydroxylated esters, for instance isostearyl lactate, octyl hydroxystearate, octyldodecyl hydroxystearate, diisostearyl malate, triisocetyl citrate and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate.

The oils may include volatile and non-volatile oils. The volatility of the solvents/oils can be determined using the evaporation speed as set forth in U.S. Pat. No. 6,338,839. The inventive compositions may contain one or more volatile silicone oils. Examples of such volatile silicone oils include linear or cyclic silicone oils having a viscosity at room temperature less than or equal to 6 centistokes (cSt) and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils that may be used include KF 96A of 6 cSt viscosity, a dimethylpolysiloxane commercial product from Shin Etsu having a flash point of 94° C. Preferably, the volatile silicone oils have a flash point of at least 40° C. Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane (L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |
| PDMS DC 200 (5 cSt) from Dow Corning | 134 | 5 |
| PDMS DC 200 (3 St) from Dow Corning | 102 | 3 |

Examples of other silicone oils that may be used in the invention include non-volatile linear polydimethylsiloxanes (PDMSs) that are liquid at room temperature; polydimethylsiloxanes that may be substituted with fluoro groups, functional groups such as hydroxyl, thiol or amine groups, aliphatic (e.g., alkyl) groups or aromatic (e.g., phenyl) groups, which are pendent and/or at the end of a silicone chain, these groups each containing from 2 to 24 carbon atoms; phenylsilicones, for instance phenyl trimethicones, phenyl dimethicones, trimethyl pentaphenyl trisiloxane, tetramethyl tetraphenyl trisiloxane, phenyl trimethylsiloxydiphenylsiloxanes (e.g., DC555 from Dow Corning), diphenyl dimethicones, diphenyl methyldiphenyl trisiloxanes and 2-phenylethyl trimethylsiloxysilicates. Other examples of silicone oils include polysiloxanes modified with fatty acids, fatty alcohols or polyoxyalkylenes, fluorosilicates and perfluoro oils.

Further, a volatile linear silicone oil may be employed in the compositions of the present invention. Suitable volatile linear silicone oils include those described in U.S. Pat. No. 6,338,839 and WO03/042221. Another example is decamethyltetrasiloxane.

The composition may contain one or more non-silicone volatile oils such as volatile hydrocarbon oils, alcohols, volatile esters and volatile ethers. Examples of such volatile non-silicone oils include volatile hydrocarbon oils having from 8 to 16 carbon atoms and their mixtures, and in particular branched C₈ to C₁₆ alkanes such as C₈ to C₁₆ isoalkanes (also known as isoparaffins), isododecane, isodecane, isohexadecane, and for example, the oils sold under the trade names of Isopar or Permethyl, the C₈ to C₁₆ branched esters such as isohexyl or isodecyl neopentanoate, and their mixtures. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| Isododecane | 43 |
| Isohexadecane | 102 |
| Isodecyl Neopentanoate | 118 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

Examples of other silicone oils that may be used in the invention include non-volatile linear polydimethylsiloxanes (PDMSs) that are liquid at room temperature; polydimethylsiloxanes that may be substituted with fluoro groups, functional groups such as hydroxyl, thiol or amine groups, aliphatic (e.g., alkyl) groups or aromatic (e.g., phenyl) groups, which are pendent and/or at the end of a silicone chain, these groups each containing from 2 to 24 carbon atoms; phenylsilicones, for instance phenyl trimethicones, phenyl dimethicones, trimethyl pentaphenyl trisiloxane, tetramethyl tetraphenyl trisiloxane, phenyl trimethylsiloxydiphenylsiloxanes (e.g., DC555 from Dow Corning), diphenyl dimethicones, diphenyl methyldiphenyl trisiloxanes and 2-phenylethyl trimethylsiloxysilicates. Other examples of silicone oils include polysiloxanes modified with fatty acids, fatty alcohols or polyoxyalkylenes, fluorosilicates and perfluoro oils.

Low viscosity oils (generally from about 5 or 10 centipoises (cps) at 25° C., and up to about 100 cps, preferably up to about 50 cps, at 25° C.), high viscosity oils (at least about 100 cps, preferably at least about 150 cps (at 25° C.) and up to about 10,000, preferably up to about 1,000 cps (at 25° C.)), and mixtures thereof can be used. Low viscosity oils are preferred. Representative of low viscosity oil (viscosity in the range of 5 to 15 cps at 25° C. is isononyl isononanoate. Other suitable low viscosity oils include, for example, octyl palmitate, diioctylmaleate, octyldedecanol, PEG-4 diheptanoate, isononylnonoate, coco-dicaprylate/caprate, polyglyceryl-3-diisostearate, cetyl alcohol, isocetyl alcohol, oleyl alcohol, cetyl acetate, acetylated lanolin alcohol, and the like. Examples of high viscosity oils (viscosities in the range of 100 to 1,000 cps at 25° C.) include lanolin oil, sesame seed oil, glyceryl trioctanoate, tridecyl trimellitate, castor oil, gcaprylic/capric triglyceride, corn oil, mineral oil, hydrogenated polyisobutene, polybutene polyvinylpyrrolidone (PVP)/hexadecene, diisoarachidyl dilinoleate, dism (diisopropyl malate) (dism) and trioctyldodecyl citrate.

Amounts of oil in the cosmetic compositions of the present invention generally ranges from about 1.0 to about 70% by weight, and in some embodiments is about 5.0 to about 40% by weight.

The compositions of the present invention may contain a structuring agent, e.g., in embodiments where the composition is mascara or a lip composition. As used herein, the term “structuring agent” refers to an agent that will at least thicken the liquid fatty phase and in some embodiments, prevent it from running between the fingers, and provides the composition as a whole with sufficient hardness so that it can be made e.g., molded, hot poured or cast into the desired shape. Where the liquid fatty phase is structured, it makes it possible to limit exudation of the fatty phase from solid compositions, and furthermore, particularly in the case of lip compositions, to limit, after deposition on the lips, its migration above and below the lip line and into the wrinkles and fine lines. Significant migration of the liquid fatty phase, laden with coloring materials, leads to an unaesthetic effect around the lips, which can accentuate the wrinkles and fine lines. This migration is often mentioned by women as being a major defect of conventional lipsticks and eyeshadows. The term “migration” is understood to mean running of the composition deposited on the lips or skin beyond its initial outline, e.g., above and below the lip line.

In some embodiments, the structuring agent includes at least one wax. For the purposes of the present invention, the term “wax” means a lipophilic fatty compound that is solid at room temperature (25° C.) and atmospheric pressure (760 mmHg, i.e. 10⁵ Pa), which undergoes a reversible solid/liquid change of state and which has a melting point of greater than 30° C. and in some embodiments, greater than 55° C. up to 120° C. or even as high as 200° C.

For the purposes of the invention, the waxes are those generally used in cosmetics and dermatology. A variety of waxes may be useful, including waxes of animal origin, waxes of plant origin, waxes of mineral origin and waxes of synthetic origin. Examples of waxes of animal origin include beeswaxes, lanolin waxes and Chinese insect waxes. Examples of waxes of plant origin include rice waxes, carnauba wax, candellila wax and ouricurry wax, cork fibre waxes, sugar cane waxes, Japan waxes, sumach wax and cotton wax. Examples of waxes of mineral origin include paraffins, microcrystalline waxes, montan waxes and ozokerites. Examples of waxes of synthetic origin include polyolefin waxes, e.g., polyethylene waxes, waxes obtained by Fischer-Tropsch synthesis, waxy copolymers and their esters, and silicone and fluoro waxes. Alternatively, hydrogenated oils of animal or plant origin may be used. Examples include hydrogenated jojoba waxes and hydrogenated oils which are obtained by catalytic hydrogenation of fats composed of a C₈-C₃₂ linear or nonlinear fatty chain, hydrogenated sunflower oil, hydrogenated castor oil, hydrogenated copra oil, hydrogenated lanolin and hydrogenated palm oils. In some embodiments, the compositions contain at least two waxes.

The wax may be present in the composition according to the invention in a content generally ranging from about 1.0% to about 50% by weight, relative to the total weight of the composition. Wax content of washable mascara can be as high as about 50% by weight, and in some embodiments, from about 10% to about 45% by weight of the composition. Wax content of waterproof mascara is slightly less e.g., up to about 40% by weight, and in some embodiments, from about 5% to about 35% by weight.

The compositions of the present invention may contain a structuring agent comprising a non-wax polymer. Examples of suitable structuring polymers are disclosed in U.S. Pat. Nos. 5,783,657 and 6,402,408. Specifically, the disclosed polymers are represented by the following formula (I): 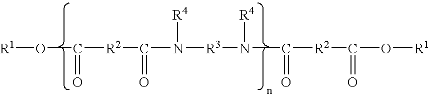
in which:

n is an integer which represents the number of amide units such that the number of ester groups present in the structuring polymer ranges from 10% to 50% of the total number of all the ester groups and all the amide groups comprised in the structuring polymer;

R¹, which are identical or different, are each chosen from alkyl groups comprising at least 4 carbon atoms and alkenyl groups comprising at least 4 carbon atoms;

R², which are identical or different, are each chosen from C₄ to C₄₂ hydrocarbon-based groups with the proviso that at least 50% of R² are chosen from C₃₀ to C₄₂ hydrocarbon-based groups;

R³, which are identical or different, are each chosen from organic groups comprising atoms chosen from carbon atoms, hydrogen atoms, oxygen atoms and nitrogen atoms with the proviso that R³ comprises at least 2 carbon atoms; and

R⁴, which are identical or different, are each chosen from hydrogen atoms, C₁ to C₁₀ alkyl groups and a direct bond to group chosen from R³ and another R⁴ such that when said at least one group is chosen from another R⁴, the nitrogen atom to which both R³ and R⁴ are bonded forms part of a heterocyclic structure defined in part by R⁴—N—R³, with the proviso that at least 50% of all R⁴ are chosen from hydrogen atoms.

In the present invention, n can be an integer ranging from 1 to 5. In the present invention, R¹, which are identical or different, can each be chosen from C₁₂ to C₂₂ alkyl groups, such as from C₁₆ to C₂₂ alkyl groups.

In the present invention, R², which are identical or different, can each be chosen from C₁₀ to C₄₂ alkyl groups. At least 50% of R², which are identical or different, can each be chosen from groups comprising from 30 to 42 carbon atoms. At least 75% of R², which are identical or different, can each be chosen from groups comprising from 30 to 42 carbon atoms. In the two aforementioned embodiments, the remaining R², which are identical or different, can each be chosen from C₄ to C₁₉ groups, such as C₄ to C₁₂ groups.

The substituent R³, which can be identical or different, can each be chosen from C₂ to C₃₆ hydrocarbon-based groups and polyoxyalkylene groups. In another example, R³, which can be identical or different, can each be chosen from C₂ to C₁₂ hydrocarbon-based groups. In another embodiment, R⁴, which can be identical or different, can each be chosen from hydrogen atoms. As used herein, hydrocarbon-based groups may be linear, cyclic or branched and saturated or unsaturated. The hydrocarbon-based groups can be aliphatic or aromatic.

Non-limiting examples the structuring polymer which may be used in the compositions of the present include the products sold by the Arizona Chemical Co. under the names Uniclear 80 and Uniclear 100. These are sold, respectively, in the form of an 80% (in terms of active material) gel in a mineral oil and a 100% (in terms of active material) gel. These polymers have a softening point ranging from 88° C. to 94° C., and may be mixtures of copolymers derived from monomers of (i) C₃₆ diacids and (ii) ethylenediamine, and have a weight-average molecular mass of about 6000. Terminal ester groups result from esterification of the remaining acid end groups with at least one alcohol chosen from cetyl alcohol and stearyl alcohol. A mixture of cetyl and stearyl alcohols is sometimes called cetylstearyl alcohol. As disclosed in the '408 patent, these polymers are advantageously used in combination with at least one amphiphilic compound which is liquid at room temperature and which has an HLB value of less than 8.

Examples of non-wax structuring polymers are also disclosed in U.S. Patent Publication no. 20050008599. Further examples are the silicone-polyamide copolymers disclosed in U.S. Patent Publication no. 20040170586.

The composition according to the invention may also contain at least one fatty compound that is pasty at room temperature. For the purposes of the invention, the expression “pasty fatty substance” means a compound with a melting point ranging from 25 to 60° C. and preferably from 30 to 45° C. and/or a hardness ranging from 0.001 to 0.5 MPa and preferably from 0.005 to 0.4 MPa.

The melting point values correspond to the melting point measured using a differential scanning calorimeter (DSC), such as the calorimeter sold under the name DSC 2920 by the company TA Instruments, with a temperature rise of 5 or 10° C., per minute. (The melting point considered is the point corresponding to the temperature of the most endothermic peak in the thermogram.)

The hardness is measured according to a method of penetration of a probe into a sample of compound and in particular using a texture analyzer (for example the TA-XT2i from Rheo) equipped with a stainless steel cylinder 2 mm in diameter. The hardness measurement is performed at 20° C. at the center of 5 samples. The cylinder is introduced into each sample at a pre-speed of 1 mm/sec and then at a measuring speed of 0.1 mm/sec, the depth of penetration being 0.3 mm. The hardness value recorded is that of the maximum peak of the applied force.

Pasty fatty substances include hydrocarbon-based compounds, optionally of polymeric type; they may also be chosen from hydrocarbon-based compounds, silicone compounds and/or fluoro compounds, and mixtures thereof. Among the pasty compounds that may be mentioned are lanolins and lanolin derivatives, for instance acetylated lanolins or oxypropylenated lanolins, with a viscosity from 18 to 21 Pa.s and preferably 19 to 20.5 Pa.s, and/or a melting point from 30 to 45° C., and mixtures thereof. Esters of fatty acids or of fatty alcohols may also be used, especially those containing 20 to 65 carbon atoms (and having a melting point of about 20 to 35° C. and/or viscosity at 40° C. ranging from 0.1 to 40 Pa.s), for instance triisostearyl or cetyl citrate; arachidyl propionate; polyvinyl laurate; cholesterol esters, for instance triglycerides of plant origin such as hydrogenated plant oils, viscous polyesters, for instance poly(12-hydroxystearic acid), and mixtures thereof. Triglycerides of plant origin that may be used include hydrogenated castor oil derivatives, such as “Thixin R” from Rheox. Mention may also be made of silicone pasty fatty substances such as polydimethylsiloxanes (PDMS) containing pendant chains of the alkyl or alkoxy type containing from 8 to 24 carbon atoms, and having a melting point of 20-55° C., for instance stearyl dimethicones, especially those sold by the company Dow Corning under the trade names DC₂₅₀₃ and DC₂-5514, and mixtures thereof.

The pasty fatty substance may be present in an amount of from 0.1 to 70% by weight, preferably from 1 to 50% by weight, and more preferably from 2 to 30% by weight, based on the weight of the composition.

In addition to water, the aqueous phase may contain a water-miscible solvent (miscibility in water of greater than 50% by weight at 25° C.), for instance, lower monoalcohols containing from 1 to 5 carbon atoms such as ethanol or isopropanol, glycols containing from 2 to 8 carbon atoms, such as propylene glycol, ethylene glycol, butylene glycol or dipropylene glycol, C3-C4 ketones and C2-C4 aldehydes. The aqueous phase may be present in a content ranging from about 1% to about 95% by weight, relative to the total weight of the composition, in some embodiments from about 3% to about 70%, 75% or 80% by weight, and more preferably from about 5% to about 45%, 50%, 55% or 60% by weight.

The compositions of the invention may contain surfactants. Surfactants typically employed in the compositions of the present invention include amphoteric, anionic, cationic and nonionic surfactants. See, e.g., Encyclopedia of Chemical Technology, KIRK-OTHMER, volume 22, pp. 333-432, 3rd edition, 1979, Wiley, for the definition of the properties and (emulsifying) functions of the surfactants, in particular pp. 347-377 of this publication regarding anionic and nonionic surfactants. Examples of surfactants useful in the compositions of the invention are include as nonionic surfactants, fatty acids, fatty alcohols, polyethoxylated fatty alcohols or polyglycerolated fatty alcohols, such as polyethoxylated stearyl alcohols or cetylstearyl alcohols, esters of fatty acid and sucrose, and glucose alkyl esters, in particular polyoxyethylenated C₁-C₆ alkyl glucose fatty esters, and as anionic surfactants, C₁₆-C₃₀ fatty acids neutralized by amines, ammonia or the alkali metal salts thereof. Examples of amphoteric surfactants include betaines, sultaines, hydroxysultaines, alkyl amphodiacetates, alkyl amphodipropionates, and imidazolines, or salts thereof. Other fatty acid condensates such as those formed with amino acids, proteins, and the like are suitable as well. Specific examples include cocamphodipropionate, e.g., Miranol C2M-SF (disodium cocamphodipropionate), in its salt-free form, available from Rhone-Poulenc, and Crosultaine C-50 (cocamidopropyl hydroxysultaine), available from Croda. Examples of cationic surfactants include quaternary amines, amine oxides and amines, e.g., alkyl amines, alkyl imidazolines, ethoxylated amines, quaternary compounds, and quaternized esters.

Surfactants are generally present in amounts ranging from about 1 to about 30% by weight, and in some other embodiments from about 5% to about 15% by weight, relative to the total weight of the composition.

Depending upon the nature of the inventive composition, controlling viscosity may be important from the standpoints of fast and easy application of the composition, as well as uniform coating. In the case of washable and waterproof mascaras, for example, viscosity of washable mascaras generally ranges from about 10 to about 60 pascal seconds (Pa.s), and preferably from about 20 to about 40 Pa.s, whereas viscosity of waterproof mascaras generally ranges from about 10 to about 70 Pa.s, and preferably from about 10 to about 40 Pa*s. Viscosity is measured at 25° C. with a Rheomat RM 180 viscometer fitted with a No. 4 rotor, wherein the measurement is carried out after spinning the rotor for 10 minutes (after which time stabilization of the viscosity and of the rotor spin speed are observed), at a shear rate of 200 s⁻¹.

Viscosity may be adjusted by adding a thickener. Representative examples include cellulose-based thickeners, for example, water-soluble cellulose-based thickeners, such as hydroxyethylcellulose, methylcellulose, hydroxypropylcellulose and carboxymethylcellulose. Among these thickeners, specific examples include alginates, maltodextrin, polysaccharide resins such as starch and its derivatives, hyaluronic acid and its salts, clays, and, in particular, montmorillonites, hectorites and laponites, crosslinked polyacrylic acids, such as the “Carbopol” products from the company Goodrich, the polyglyceryl (meth)acrylate polymers sold under the names “Hispagel” or “Lubragel” by the companies Hispano Quimica or Guardian, polyvinylpyrrolidone (PVP), polyvinyl alcohol, crosslinked acrylamide polymers and copolymers, such as those sold under the names “PAS 5161” or “Bozepol C” by the company Hoechst, “Sepigel 305” by the company SEPPIC, crosslinked methacryloyloxyethyltrimethylammonium chloride homopolymers sold under the name “Salcare SC₉₅” by the company Allied Colloid, and associative polymers and, in particular, associative polyurethanes. Oil soluble thickening agents may also be used. See, U.S. Patent Publications 2003/0215413, 2005/0065046 and 2002/0028226.

The thickening agent is generally present in an amount ranging from about 0.05% to about 20% by weight, and preferably from about 0.5% to about 10% by weight.

The compositions of the present invention may additionally contain a film-forming polymer, which may be an organic or inorganic polymer. In one embodiment, the film-forming organic polymer is at least one polymer chosen from the group comprising:
liposoluble film-forming polymers,
lipodispersible film-forming polymers in the form of non-aqueous dispersions of polymer particles, preferably dispersions in silicone or hydrocarbon oils; in one embodiment, the non-aqueous dispersions of polymer comprise polymer particles stabilised on their surface by at least one stabilising agent; these non-aqueous dispersions are often called “NAD”,
aqueous dispersions of polymer particles, often called “latex”; in that case, the composition must comprise an aqueous phase,
water-soluble film-forming polymers; in that case, the composition must comprise an aqueous phase.

In one embodiment, the film-forming agent is a film-forming liposoluble or lipodispersible organic polymer.

I/ Liposoluble Polymers

They have no, or very little, gelling nature for the oil medium chosen, in particular when they are used at a concentration less than or equal to 50% by weight. The liposoluble polymers may be of any chemical type and include especially:
a) the liposoluble, amorphous homopolymers and copolymers of olefins, cycloolefins, butadiene, isoprene, styrene, ethers, vinyl esters or amides, esters or amides of (meth)acrylic acid containing a linear, branched or cyclic C₄₋₅₀ alkyl group, and preferably amorphous. The preferred liposoluble homopolymers and copolymers are obtained from monomers chosen from within the group consisting of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tertio-butyl (meth)acrylate, tridecyl (meth)acrylate, stearyl (meth)acrylate, or mixtures thereof. There will be cited, for example, the alkyl acrylate/cycloalkyl acrylate copolymer sold by PHOENIX CHEM. under the name GIOVAREZ AC-5099 ML, and the vinyl pyrrolidone homo- and copolymers, such as copolymers of a C₂ to C₃₀, such as C₃ to C₂₂ alkene, and combinations thereof, can be used. As examples of PVP and VP copolymers which can be used in the invention, mention may be made of PVP/eicosene, VP/vinyllaurate, VP/vinylstearate butylated polyvinylpyrrolidone (PVP), VP/hexadecene, VP/triacontene or VP/acrylic acid/lauryl methacrylate copolymer.

As special liposoluble copolymers, there may be cited:
i) the grafted acrylic-silicone polymers with a silicone backbone, acrylic grafts or with an acrylic backbone, silicone grafts such as the product sold under the name SA 70.5 by 3M and described in U.S. Pat. Nos. 5,725,882, 5,209,924, 4,972,037, 4,981,903, 4,981,902, 5,468,477 and 5,219,560, and in EP 0 388,582.
ii) the liposoluble polymers bearing fluorous groups belonging to one of the classes described hereinabove, in particular those described in the U.S. Pat. No. 5,948,393, the alkyl (meth)acrylate/perfluoroalkyl (meth)acrylate copolymers described in the EP 0 815,836 and U.S. Pat. No. 5,849,318.
iii) polymers or copolymers resulting from the polymerization or copolymerization of an ethylenic monomer, comprising one or more ethylenic, preferably conjugated bonds (or dienes). As polymers or copolymers resulting from the polymerization or copolymerization of an ethylenic monomer, use may be made of vinyl, acrylic or methacrylic copolymers which may be block copolymers, such as diblock or triblock copolymers, or even multiblock or starburst or radial copolymers. The at least one ethylenic film-forming agent may comprise, for example, a styrene block (S), an alkylstyrene block (AS), an ethylene/butylene block (EB), an ethylene/propylene block (EP), a butadiene block (B), an isoprene block (I), an acrylate block (A), a methacrylate block (MA) or a combination of these blocks.

In one embodiment, a copolymer comprising at least one styrene block is used as film-forming agent. A triblock copolymer and in particular those of the polystyrene/polyisoprene or polystyrene/ polybutadiene type, such as those sold or made under the name “Luvitol HSB” by BASF and those of the polystyrene/copoly(ethylene-propylene) type or alternatively of the polystyrene/copoly(ethylene/butylene) type, such as those sold or made under the brand name “Kraton” by Shell Chemical Co. or Gelled Permethyl 99A by Penreco, may be used. Styrene-methacrylate copolymers can also be used.

As ethylenical film-forming agent which can be used in the composition of the invention, mention may be made, for example, of Kraton (G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton G1750X (EP) multiarm, Kraton G1765X (EP) multiarm, Kraton D-1101 (SBS), Kraton D-1102 (SBS), Kraton D-1107 (SIS), Gelled Permethyl 99A-750, Gelled Permethyl 99A-753-58 (mixture of starburst block polymer and triblock polymer), Gelled Permethyl 99A-753-59 (mixture of starburst block polymer and triblock polymer), Versagel 5970 and Versagel 5960 from Penreco (mixture of starburst polymer and triblock polymer in isododecane), and OS 129880, OS 129881 and OS 84383 from Lubrizol (styrene-methacrylate copolymer). As examples of liposoluble polymers that can be used in the invention, mention may be made of polyalkylenes, in particular polybutene.
b) the liposoluble, amorphous polycondensates, preferably not comprising hydrogen-interaction donor groups, in particular polyesters with C₄₋₅₀ alkyl side chains or else polyesters resulting from the condensation of fatty acid dimers, or even polyesters comprising a silicone segment in the form of a sequence, graft or end group, solid at room temperature; and
c) the liposoluble, amorphous polysaccharides comprising alkyl (ether or ester) side chains, in particular alkylcelluloses with a linear or branched, saturated or unsaturated C₁ to C₈ alkyl radical, such as ethylcellulose and propylcellulose.

As a general rule, the film-forming liposoluble polymers of the invention have a molecular weight ranging between 1,000 and 500,000, preferably between 2,000 and 250,000, and a glass transition temperature ranging between −100° C. and +300° C., preferably between −50° C. and +100° C., preferably still between −10° C. and +90° C.

II/ Lipodispersible Polymers: Non-aqueous Dispersions of Polymer Particles

According to another embodiment, the at least one film-forming polymer can be chosen from stable non-aqueous dispersions of polymer particles, that are generally spherical, of one or more polymers, in a physiologically acceptable liquid fatty phase, such as hydrocarbon-based oils or silicone oils. These dispersions are generally known as NADs (non-aqueous dispersions) of polymer, as opposed to lattices, which are aqueous dispersions of polymer. These dispersions may especially be in the form of nanoparticles of polymers in stable dispersion in the said fatty phase. In one embodiment, the nanoparticles are between 5 nm and 600 nm in size. However, it is possible to obtain polymer particles ranging up to 1 μm in size.

One advantage of the polymer dispersion of the composition of the invention is the possibility of varying the glass transition temperature (Tg) of the polymer or the polymer system (polymer plus additive of the plasticizer type), and of thus going from a hard polymer to a more or less soft polymer, making it possible to adjust the mechanical properties of the composition depending on the intended application and in particular on the film deposited.

The polymers in dispersion which may be used in the composition of the invention preferably have a molecular weight of about from 2,000 to 10,000,000 and a Tg of from −100° C., to 300° C. and better still from −50° C. to 50° C. and preferably from −10° C. to 100° C.

It is possible to use film-forming polymers, that preferably have a low Tg, of less than or equal to the temperature of the skin and especially less than or equal to 40° C. A dispersion is thus obtained which can form a film when it is applied to a support.

Among the film-forming polymers which may be mentioned are free-radical, acrylic or vinyl homopolymers or copolymers, preferably having a Tg of less than or equal to 40° C. and especially ranging from −10° C. to 30° C., used alone or as a mixture.

The expression “free-radical polymer” as used herein, refers to a polymer obtained by polymerization of monomers containing unsaturation, especially ethylenic unsaturation, each monomer being capable of homopolymerizing (unlike polycondensates). The free-radical polymers may especially be vinyl polymers or copolymers, especially acrylic polymers.

The vinyl polymers may result from the polymerization of ethylenically unsaturated monomers containing at least one acid group and/or esters of these acidic monomers and/or amides of these acids.

As monomers bearing an acidic group, it is possible to use α,β-ethylenic unsaturated carboxylic acids such as acrylic acid, methacrylic acid, crotonic acid, maleic acid or itaconic acid. (Meth)acrylic acid and crotonic acid are preferably used, and more preferentially (meth)acrylic acid.

The esters of acidic monomers are advantageously chosen from the esters of (meth)acrylic acid (also known as (meth)acrylates), for instance alkyl (meth)acrylates, in particular of a C₁-C₂₀ and preferably a C₁-C₈ alkyl, aryl (meth)acrylates, in particular of a C₆-C₁₀ aryl, and hydroxyalkyl (meth)acrylates, in particular of a C₂-C₆ hydroxyalkyl. Alkyl (meth)acrylates which may be mentioned include methyl, ethyl, butyl, isobutyl, 2-ethylhexyl and lauryl (meth)acrylate. Hydroxyalkyl (meth)acrylates which may be mentioned include hydroxyethyl (meth)acrylate and 2-hydroxypropyl (meth)acrylate. Aryl (meth)acrylates which may be mentioned include benzyl or phenyl acrylate.

The (meth)acrylic acid esters that are particularly preferred are the alkyl (meth)acrylates.

Free-radical polymers that are preferably used are copolymers of (meth)acrylic acid and of an alkyl (meth)acrylate, especially of a C₁-C₄ alkyl. More preferentially, methyl acrylates may be used, optionally copolymerized with acrylic acid.

Amides of the acidic monomers which may be mentioned include (meth)acrylamides, and especially N-alkyl(meth)acrylamides, in particular of a C₂-C₁₂ alkyl, such as N-ethylacrylamide, N-t-butylacrylamide and N-octylacrylamide; N-di(C₁-C₄)alkyl (meth)acrylamides.

The vinyl polymers may also result from the polymerization of ethylenically unsaturated monomers containing at least one amine group, in free form or partially or totally neutralized, or alternatively partially or totally quaternized. Such monomers may be, for example, dimethylaminoethyl (meth)acrylate, dimethylaminoethylmethacrylamide, vinylamine, vinylpyridine and diallyldimethylammonium chloride.

The vinyl polymers may also result from the homopolymerization or copolymerization of at least one monomer chosen from vinyl esters and styrene monomers. In particular, these monomers may be polymerized with acidic monomers and/or esters thereof and/or amides thereof, such as those mentioned above. Examples of vinyl esters which may be mentioned include vinyl acetate, vinyl propionate, vinyl neodecanoate, vinyl pivalate, vinyl benzoate and vinyl t-butylbenzoate. Styrene monomers, which may be mentioned, include styrene and α-methylstyrene.

The list of monomers given is not limiting, and it is possible to use any monomer known to those skilled in the art that falls within the categories of acrylic and vinyl monomers (including monomers modified with a silicone chain). As other vinyl monomers that may be used, mention may also be made of:
N-vinylpyrrolidone, vinylcaprolactam, vinyl-N-(C₁-C₆)-alkylpyrroles, vinyloxazoles, vinylthiazoles, vinyl-pyrimidines and vinylimidazoles,
olefins such as ethylene, propylene, butylene, isoprene and butadiene.

The vinyl polymer may be partially crosslinked with one or more difunctional monomers, especially comprising at least two ethylenic unsaturations, such as ethylene glycol dimethacrylate or diallyl phthalate.

In a non-limiting manner, the polymers in dispersion of the invention may be chosen from the following polymers or copolymers: polyurethanes, polyurethane-acrylics, polyureas, polyurea-polyurethanes, polyester-polyurethanes, polyether-polyurethanes, polyesters, polyesteramides, fatty-chain polyesters, alkyds; acrylic and/or vinyl polymers or copolymers; acrylic-silicone copolymers; polyacrylamides; silicone polymers, for instance silicone polyurethanes or silicone acrylics, and fluoro polymers and mixtures thereof.

The polymer(s) in oily dispersion may represent (as solids or active material) from 0.1% to 60% of the weight of the composition, preferably from 2% to 40% and better still from 4% to 25%. For a stabilizer that is solid at ambient temperature, the amount of solids in the dispersion represents the total amount of polymer and stabilizer.

The liposoluble or dispersible polymers in the composition of the invention can be also used in an amount of from 0.01% to 20% (as active material) relative to the total weight of the composition, such as, for example, from 1% to 10%, if they are present.

III) Aqueous Dispersions of Polymer Particles

According to another embodiment, the at least one film-forming polymer can be chosen from aqueous dispersions of polymer particles, in case the composition according to the invention comprises an aqueous phase.

The aqueous dispersion comprising one or more film-forming polymers may be prepared by the person skilled in the art in particular by emulsion polymerization or by placement in dispersion of the previously formed polymer.

Among the film-forming polymers that may be used in the composition according to this invention, there may be cited synthetic polymers of the polycondensate type or the radical type, polymers of natural origin, and mixtures thereof.

Among the polycondensates, there also may be cited the anionic, cationic, nonionic or amphoteric polyurethanes, polyurethane-acrylics, polyurethane-polyvinylpyrrolidones, polyester-polyurethanes, polyether-polyurethanes, polyureas, polyurea/polyurethanes, and mixtures thereof.

The polyurethanes may be, for example, an aliphatic, cycloaliphatic or aromatic polyurethane, polyurea/urethane or polyurea copolymer comprising, alone or as a mixture:
at least one sequence of linear or branched aliphatic and/or cycloaliphatic and/or aromatic polyester origin, and/or
at least one sequence of aliphatic and/or cycloaliphatic and/or aromatic polyether origin, and/or
at least one silicone sequence, substituted or unsubstituted, branched or unbranched, for example polydimethylsiloxane or polymethylphenylsiloxane, and/or
at least one sequence comprising fluorous groups.

The polyurethanes such as defined in the invention also may be obtained from branched or unbranched polyesters or from alkyds comprising mobile hydrogens which are modified by means of polyaddition with a diisocyanate and an organic bifunctional co-reactive (for example dihydro, diamino or hydroxyamino) compound, further comprising either a carboxylate or carboxylic acid group, or a sulfonic acid or sulfonate group, or even a neutralizable tertiary amine group or a quaternary ammonium group.

There also may be cited the polyesters, polyester amides, polyesters with a fatty chain, polyamides, and epoxyester resins.

The polyesters may be obtained in known manner by means of polycondensation of aliphatic or aromatic diacids with aliphatic or aromatic diols or with polyols. Succinic acid, glutaric acid, adipic acid, pimelic acid, suberic acid or sebacic acid may be used as aliphatic diacids. Terephthalic acid or isophthalic acid, or else even a derivative such as phthalic anhydride, may be used as aromatic diacids. Ethylene glycol, propylene glycol, diethylene glycol, neopentyl glycol, cyclohexane dimethanol, 4,4N-(1-methylpropylidene)bisphenol, may be used as aliphatic diols. Glycerol, pentaerythritol, sorbitol, and trimethylol propane may be used as polyols.

The polyester amides may be obtained in a manner analogous to the polyesters, by means of polycondensation of diacids with diamines or amino alcohols. Ethylene diamine, hexamethylenediamine, meta- or paraphenylene diamine may be used as diamine. Monoethanolamine may be used as amino alcohol.

As a monomer bearing an anionic group which may be used during polycondensation, there may be cited, for example, dimethylol propionic acid, trimellitic acid or a derivative such as trimellitic anhydride, the sodium salt of 3-sulfo pentanediol acid, the sodium salt of 5-sulfo 1,3-benzenedicarboxylic acid. Polyesters with a fatty chain may be obtained through the use of diols with a fatty chain during polycondensation. The epoxyester resins may be obtained by polycondensation of fatty acids with a condensate at the α,ω-diepoxy ends.

The radical-type polymers may be in particular acrylic and/or vinyl polymers or copolymers. Anionic radical polymers preferably are used. As a monomer bearing an anionic group that may be used during radical polymerization, there may be cited acrylic acid, methacrylic acid, crotonic acid, maleic anhydride, and 2-acrylamido 2-methyl propane sulfonic acid.

The acrylic polymers may result from the copolymerization of monomers chosen from among the esters and/or amides of acrylic acid or methacrylic acid. As examples of ester-type monomers, there may be cited methyl methacrylate, ethyl methacrylate, butyl methacrylate, isobutyl methacrylate, 2-ethyl hexyl methacrylate, lauryl methacrylate. As examples of amide-type monomers, there may be cited N-t-butyl acrylamide and N-t-octyl acrylamide.

There preferably are used acrylic polymers obtained by copolymerization of monomers with ethylene unsaturation containing hydrophilic groups, preferably of a nonionic nature, such as hydroxyethyl acrylate, 2-hydroxypropyl acrylate, hydroxyethyl methacrylate, and 2-hydroxypropyl methacrylate.

The vinyl polymers may result from the homopolymerization or from the copolymerization of monomers chosen from among the vinyl esters, styrene, or butadiene. As examples of vinyl esters, there may be cited vinyl acetate, vinyl neodecanoate, vinyl pivalate, vinyl benzoate and vinyl t-butyl benzoate.

Acrylic/silicone copolymers or even nitrocellulose/acrylic copolymers also may be used.

Polymers of natural origin, possibly modified, may be chosen from among shellac resin, the dammars, elemis, copals, cellulosic derivatives, and mixtures thereof.

There also may be cited the polymers resulting from the radical polymerization of one or more radical monomers, inside and/or partially on the surface, of preexisting particles of at least one polymer chosen from among the group consisting of polyurethanes, polyureas, polyesters, polyester amides and/or alkyds. These polymers generally are called hybrid polymers.

When an aqueous dispersion of polymer particles is used, the dry-matter content of said aqueous dispersion may be on the order of 5-60% by weight, and preferably 30-50%.

The size of the polymer particles in aqueous dispersion may range between 10-500 nm, and preferably ranges between 20 and 150 nm, which makes it possible to obtain a film having a notable sheen. Particle sizes ranging up to one micron, however, may be used.

IV) Water-soluble Polymers

In some embodiments, the film-forming polymer may be a water-soluble polymer and is therefore present in the aqueous phase of the composition in solubilized form. Among the water-soluble film-forming polymers, the following cationic polymers may be cited:
(1) acrylic polymers or copolymers such as polyacrylates or polymethacrylates;

The copolymers of family (1) furthermore may contain one or more moieties deriving from comonomers which may be chosen from within the family of acrylamides, methacrylamides, diacetone acrylamides, acrylamides and methacrylamides substituted on nitrogen with lower alkyls, acrylic or methacrylic acids or esters thereof, vinylactams such as vinylpyrrolidone or vinylcaprolactam, vinyl esters.

Thus, among these copolymers of family (1), there may be cited:
the copolymers of acrylamide and dimethylaminoethyl methacrylate quaternized with dimethyl sulfate, or with a dimethyl halogenide such as that sold under the name HERCOFLOC by the company HERCULES,
the copolymers of acrylamide and methacryloyloxyethyltrimethylammonium chloride described, for example, in patent application EP-A-080976,
the copolymer of acrylamide and methacryloyloxyethyltrimethylammonium methosulfate sold under the name RETEN by the company HERCULES,
the vinylpyrrolidone/dialkylaminoalkyl acrylate or methacrylate copolymers, quaternized or non-quaternized, such as the products sold under the name “GAFQUAT” by the company ISP such as, for example, “GAFQUAT 734” or “GAFQUAT 755” or else the products designated as “COPOLYMER 845, 958 and 937.1” These polymers are described in French patents 2,077,143 and 2,393,573,
the dimethylaminoethyl methacrylate/vinylcaprolactam/vinylpyrrolidone terpolymers such as the product sold under the name GAFFIX VC 713 by the company ISP, and
the quaternized vinylpyrrolidone/dimethylaminopropyl methacrylamide copolymer such as the product sold under the name “GAFQUAT HS 100” by the company ISP.
(2) the quaternary vinylpyrrolidone and vinylimidazole copolymers;
(3) the chitosans or salts thereof;
(4) the cationic cellulose derivatives such as copolymers of cellulose or cellulose derivatives grafted with a water-soluble monomer comprising a quaternary ammonium and described in particular in U.S. Pat. No. 4,131,576, such as the hydroxyalkyl celluloses, like the hydroxymethyl, hydroxyethyl or hydroxypropyl celluloses grafted in particular with a methacryloyloxyethyl trimethylammonium, methacrylamidopropyl trimethylammonium, dimethyl-diallylammonium salt. The marketed products corresponding to this definition are most especially the products sold under the name “CELQUAT L 200” and CELQUAT H 100” by the National Starch Company.

Among the film-forming water-soluble polymers, the following amphoteric polymers may be cited:
(1) the polymers resulting from the copolymerization of a monomer derived from a vinyl compound bearing a carboxylic group such as most especially acrylic acid, methacrylic acid, maleic acid, alpha-chloracrylic acid, and of a basic monomer derived from a substituted vinyl compound containing at least one base atom such as most particularly dialkylaminoalkylmethacrylate and acrylate, dialkylaminoalkylmethacrylamide and acrylamide. Such compounds are described in U.S. Pat. No. 3,836,537.
(2) the polymers comprising moieties deriving:
   a) from at least one monomer chosen from among the acrylamides or methacrylamides substituted on nitrogen with an alkyl radical,
   b) from at least one acid comonomer containing one or more reactive carboxylic groups, and
   c) from at least one basic comonomer such as esters, with primary, secondary, tertiary and quaternary amine substituents, of acrylic and methacrylic acids, and the product of quaternization of dimethylaminoethyl methacrylate with dimethyl or diethyl sulfate.
(3) the crosslinked alkoyl polyamino amides deriving in whole or in part from polyaminoamides
(4) the polymers comprising zwitterionic moieties
(5) the polymer derived from chitosan
(6) the polymers derived from N-carboxyalkylation of chitosan, such as N-carboxymethyl chitosan or N-carboxybutyl chitosan sold under the name “EVALSAN” by the company JAN DEKKER.
(7) the copolymers of alkyl(C1-C5)vinylether/maleic anhydride partially modified by semiamidification with an N,N-dialkylaminoalkylamine such as N,N-dimethylaminopropylamine or by semiesterification with an N,N-dialcanolamine, These copolymers also may comprise other vinyl comonomers such as vinylcaprolactam.

Water-soluble film-forming polymers are preferably selected from:
proteins such as proteins of plant origin, such as wheat or soya bean proteins; proteins of animal origin such as keratin, for example keratin hydrolysates and sulphonic keratins;
anionic, cationic, amphoteric or nonionic polymers of chitin or chitosan;
cellulose polymers such as hydroxyethyl cellulose, hydroxypropyl cellulose, methyl cellulose, ethyl hydroxyethyl cellulose, carboxymethyl cellulose, and quaternized derivatives of cellulose;
acrylic polymers or copolymers such as polyacrylates or polymethacrylates;
vinyl polymers, such as polyvinylpyrrolidones, copolymers of methyl vinyl ether and maleic anhydride, the copolymer of vinyl acetate and crotonic acid, copolymers of vinylpyrrolidone and vinyl acetate; copolymers of vinylpyrrolidone and caprolactam; and polyvinyl alcohols;

These polymers will be used in particular if a more or less appreciable elimination of the film with water is desired.

In order to improve the film-forming nature of an oily or aqueous polymer, it is possible to add to the polymeric system a coalescent agent which may be chosen from among the known coalescent agents.

According to other embodiments of the invention, the film-forming polymer is chosen from among the polymers with a non-silicone organic backbone grafted with monomers containing a polysiloxane. These polymers may be liposoluble, lipodispersible, water-soluble or dispersible in an aqueous medium, as the case may be.

The polymers with a non-silicone organic backbone grafted with monomers containing a polysiloxane consist of a main organic chain formed from organic monomers not comprising silicone, on which there is grafted, within said chain as well as possibly on at least one of the ends thereof, at least one polysiloxane macromer.

In the following, “polysiloxane macromer” is understood to denote, as generally accepted, any monomer containing a polysiloxane-type polymer chain in its structure.

The non-silicone organic monomers making up the main chain of the grafted silicone polymer may be chosen from among the monomers with ethylene unsaturation which are polymerizable by the radical method, monomers polymerizable by polycondensation such as those forming polyamides, polyesters, polyurethanes, cycle-opening monomers such as those of the oxazoline or caprolactone type.

The polymers with a non-silicone organic backbone grafted with monomers containing a polysiloxane according to this invention may be obtained in accordance with any method known to the person skilled in the art, in particular by reaction between (i) a starting polysiloxane macromer correctly functionalized on the polysiloxane chain and (ii) and one or more non-silicone organic compounds, themselves correctly functionalized with a function which is capable of reacting with the functional group or groups borne by said silicone by forming a covalent bond; a classic example of such a reaction is the radical reaction between a vinyl group borne on one of the ends of the silicone with a double bond of a monomer with ethylene unsaturation of the main chain.

The polymers with a non-silicone organic backbone grafted with monomers containing a polysiloxane according to the invention preferably are chosen from among those described in U.S. Pat. Nos. 4,693,935, 4,728,571 and 4,972,037, and patent publications EP-A-0 412,704, EP-A-0 412,707, EP-A-0 640,105 and WO 95/00578. It concerns copolymers obtained by radical polymerization starting from monomers with ethylene unsaturation and silicone monomers having a vinyl end group or else copolymers obtained by reaction of a polyolefin containing functionalized groups and a polysiloxane macromer having an end function reactive with the functionalized groups.

A family of grafted silicone polymers suitable for use in the invention consists of the grafted silicone polymers containing:
a) from 0 to 98% by weight of at least one lipophilic monomer (A) of low lipophilic polarity with ethylene unsaturation, polymerizable by the radical method;
b) from 0 to 98% by weight of at least one hydrophilic polar monomer (B) with ethylene unsaturation, copolymerizable with the monomer or monomers of type (A);
c) from 0.01 to 50% by weight of at least one polysiloxane macromer (C) of general formula:
   X(Y)ₙSi(R)₃₋ₘZₘ (II)
in which:
   X designates a vinyl group copolymerizable with the monomers (A) and (B);
Y designates a group with a divalent bond;
   R designates a hydrogen, a C₁-C₆ alkyl or alkoxy, a C₆-C₁₂ aryl;
   Z designates a monovalent polysiloxane moiety having an average molecular weight by number of at least 500;
   n is 0 or 1 and m is an integer ranging from 1 to 3; the percentages being calculated in relation to the total weight of the monomers (A), (B) and (C).

These polymers have an average molecular weight by number ranging from 10,000 to 2,000,000 and preferably a glass transition temperature Tg or a crystalline melting temperature Tm of at least −20° C.

As examples of lipophilic monomers (A) there may be cited C₁-C₁₈ alcohol esters of acrylic or methacrylic acid; styrene; polystyrene macromers; vinyl acetate; vinyl propionate; alpha-methylstyrene; tertiobutylstyrene; butadiene; cyclohexadiene; ethylene; propylene; vinyltoluene; esters of acrylic or methacrylic acid and of 1,1-dyhydroperfluoroalkanols or homologs thereof; esters of acrylic or methacrylic acid and omega-hydrydofluoralkanols; esters of acrylic or methacrylic acid and fluoroalkylsulfoamido-alcohols; esters of acrylic or methacrylic acid and fluoroalkyl alcohols; esters of acrylic or methacrylic acid and alcohol fluoroethers; or mixtures thereof. The preferred monomers (A) are chosen from within the group consisting of n-butyl methacrylate, isobutyl methacrylate, tertio-butyl acrylate, tertio-butyl methacrylate, 2-ethylhexyl methacrylate, methyl methacrylate, 2-(N-methyl perfluoro-octane sulfonamido)-ethylacrylate; 2-(N-butylperfluorooctane sulfonamido)-ethylacrylate or mixtures thereof.

As examples of polar monomers (B) there may be cited acrylic acid, methacrylic acid, N,N-dimethylacrylamide, dimethylaminoethyl methacrylate, quaternized dimethylaminoethyl methacrylate, (meth)acrylamide, N-t-butyl acrylamide, maleic acid, maleic anhydride and demi-esters thereof, hydroxyalkyl (meth)acrylates, diallyldimethylammonium chloride, vinyl-pyrrolidone, vinyl ethers, maleimides, vinylpyridine, vinylimidazole, heterocyclic vinyl polar compounds, styrene sulfonate, allyl alcohol, vinyl alcohol, vinyl caprolactam or mixtures thereof. The monomers (B) preferably are chosen from within the group consisting of acrylic acid, N,N-dimethylacrylamide, dimethylaminoethyl methacrylate, quaternized dimethylaminoethyl methacrylate, vinylpyrrolidone and mixtures thereof.

The polysiloxane macromers (C) of formula (II) preferably are chosen from among those corresponding to the following general formula (III): 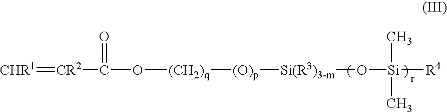
in which:
R¹ is hydrogen or —COOH (preferably hydrogen);
R² is hydrogen, methyl or —CH₂COOH (preferably methyl);
R³ is C₁-C₆ alkyl, alkoxy or alkylamino, C₆-C₁₂ aryl or hydroxyl (preferably methyl);
R⁴ is C₁-C₆ alkyl, alkoxy or alkylamino, C₆-C₁₂ aryl or hydroxyl (preferably methyl);
q is an integer from 2 to 6 (preferably 3);
p is 0 or 1;
r is a whole number from 5 to 700;
m is an integer ranging from 1 to 3 (preferably 1);

There preferably are used polysiloxane macromers of formula: 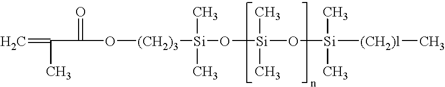
n being a number ranging from 5 to 700 and 1 being an integer ranging between 0 and 3.

One embodiment of the invention consists in using a copolymer able to be obtained by radical polymerization starting from the mixture of monomers consisting of:

a) 60% by weight of tertiobutyl acrylate;

b) 20% by weight of acrylic acid;

c) 20% by weight of silicone macromer of formula: 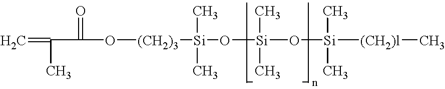
with n being a number ranging from 5 to 700 and 1 being an integer ranging between 0 and 3, the percentages by weight being calculated in relation to the total weight of the monomers.

Another embodiment of the invention entails use of a copolymer able to be obtained by radical polymerization starting from the mixture of monomers consisting of:

a) 80% by weight of tertiobutyl acrylate;

b) 20% by weight of silicone macromer of formula: 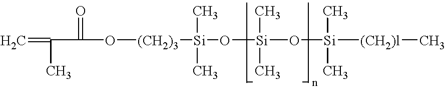
with n being a number ranging from 5 to 700 and 1 being an integer ranging between 0 and 3, the percentages by weight being calculated in relation to the total weight of the monomers.

Another special family of grafted silicone polymers with a non-silicone organic backbone suitable for use in the present invention consists of the grafted silicone copolymers able to be obtained by reactive extrusion of a polysiloxane macromer having an end reactive function on a polymer of the polyolefin type comprising reactive groups capable of reacting with the end function of the polysiloxane macromer to form a covalent bond allowing for the grafting of the silicone on the main chain of the polyolefin. These polymers, as well as the process for preparation thereof, are described in International patent application WO 95/00578.

The reactive polyolefins preferably are chosen from among the polyethylenes or the polymers of monomers derived from ethylene such as propylene, styrene, alkyl styrene, butylene, butadiene, the (meth)acrylates, the vinyl esters or equivalents, comprising reactive functions capable of reacting with the end function of the polysiloxane macromer. They are chosen most particularly from among the copolymers of ethylene or ethylene derivatives and monomers chosen from among those comprising a carboxylic function such as (meth)acrylic acid; those comprising an acid anhydride function such as maleic acid anhydride; those comprising an acid chloride function such as (meth)acrylic acid chloride; those comprising an ester function such as the esters of (meth)acrylic acid; those comprising an isocyanate function.

The silicone macromers preferably are chosen from among the polysiloxanes comprising a functionalized group, at the end of the polysiloxane chain or near the end of the chain, chosen from within the group consisting of alcohols, thiols, epoxy, primary and secondary amines, and most particularly from among those corresponding to the general formula:
T-(CH₂)ₛ—Si—[—(OSiR⁵R⁶)ₜ—R⁷]_{y} (IV)
in which T is chosen from within the group consisting of NH₂, NHRN, an epoxy function, OH, SH; R⁵, R⁶, R⁷ and RN, independently, denote a C₁-C₆ alkyl, a C₆-C₁₂ phenyl, benzyl or alkylphenyl, hydrogen; s is a number from 2 to 100, t is a number from 0 to 1000 and y is a number from 1 to 3. They have an average molecular weight by number preferably ranging from 5,000 to 300,000, more preferably from 8,000 to 200,000 and more particularly from 9,000 to 40,000.

According to an embodiment, the film-forming polymer may be purchased from Minnesota Mining and Manufacturing Company under the trade names “Silicone Plus” polymers. For example, poly(isobutyl methacrylate-co-methyl FOSEA)-g-poly(dimethylsiloxane) is sold under the trade name SA 70-5 IBMMF.

According to another embodiment of this invention, the film-forming polymer is chosen from among the silicone polymers grafted with non-silicone organic monomers. These polymers may be liposoluble, lipodispersible, water-soluble or dispersible in an aqueous medium, as the case may be.

The grafted silicone polymer or polymers, with a polysiloxane backbone grafted with non-silicone organic monomers containing a main silicone (or polysiloxane (/Si—O—)ₙ) chain on which there is grafted, within said chain as well as possibly on at least one of its ends, at least one organic group not comprising silicone.

The polymers with a polysiloxane backbone grafted with non-silicone organic monomers according to the invention may be existing commercial products or else obtained according to any means known to the person skilled in the art, in particular by reaction between (i) a starting silicone correctly functionalized on one or more of these silicon atoms and (ii) a non-silicone organic compound itself correctly functionalized with a function which is capable of reacting with the functional group or groups borne by said silicone by forming a covalent bond; a classic example of such a reaction is the hydroxsylilation reaction between /Si—H groups and CH₂═CH— vinyl groups, or else the reaction between —SH thio-functional groups with these same vinyl groups. See, U.S. Pat. No. 6,503,494.

Examples of polymers with a polysiloxane backbone grafted with non-silicone organic monomers suitable for use in this invention, as well as the specific method of preparation thereof, are described in particular in patent applications EP-A-0 582,152, WO 93/23009 and WO 95/03776.

According to an embodiment of this invention, the silicone polymer, with a polysiloxane backbone grafted with non-silicone organic monomers which is utilized is composed of the result of radical copolymerization between, on the one hand, at least one non-silicone anionic organic monomer having an ethylene unsaturation and/or a non-silicone hydrophobic organic monomer having an ethylene unsaturation and, on the other, a silicone having in its chain at least one, and preferably several, functional groups capable of reacting on said ethylene unsaturations of said non-silicone monomers by forming a covalent bond, in particular thio-functional groups.

The anionic monomers with ethylene unsaturation preferably are chosen, alone or as a mixture, from among the linear or branched unsaturated carboxylic acids, possibly neutralized in whole or in part in the form of a salt, the unsaturated carboxylic acid(s) being most particularly acrylic acid, methacrylic acid, maleic acid, maleic anhydride, itaconic acid, fumaric acid and crotonic acid. The suitable salts are in particular alkaline, alkaline earth and ammonium salts. It likewise will be noted that, in the final grafted silicone polymer, the organic group with an anionic nature which is composed of the result of radical (homo)polymerization of at least one unsaturated carboxylic-acid-type anionic monomer may be, after reaction, post-neutralized with a base (soda, ammonia, etc.) to bring it to the form of a salt.

According to this invention, the hydrophobic monomers with ethylene unsaturation preferably are chosen, alone or as a mixture, from among the alkanol acrylic acid esters and/or the alkanol methacrylic acid esters. The alkanols preferably are C₁-C₁₈ and more particularly C₁-C₁₂. The preferred monomers are chosen from within the group consisting of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tertio-butyl (meth)acrylate, tridecyl (meth)acrylate, stearyl (meth)acrylate or mixtures thereof.

A family of silicone polymers with a polysiloxane backbone grafted with non-silicone organic monomers particularly well suited for implementation of this invention consists of silicone polymers comprising in their structure the moiety of formula V below: 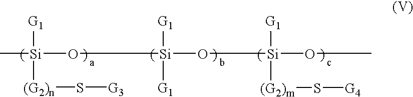
in which the radicals G₁, identical or different, represent hydrogen or a C₁-C₁₀ alkyl radical or even a phenyl radical; the radicals G₂, identical or different, represent represents a C₁-C₁₀ alkylene group; G₃ represents a polymeric residue resulting from (homo)polymerization of at least one anionic monomer with ethylene unsaturation; G₄ represents a polymeric residue resulting from (homo)polymerization of at least one monomer of at least one hydrophobic monomer with ethylene unsaturation; m and n are equal to 0 or 1; a is a whole number ranging from 0 to 50; b is a whole number which may range between 10 and 350; and c is a whole number ranging from 0 to 50, on condition that one of the parameters a and c is other than 0.

The moiety of formula (V) hereinabove preferably has at least one, and more preferably still, all of the following characteristics:
the G₁ radicals denote an alkyl radical, preferably the methyl radical;
n is not zero, and the G₂ radicals represent a divalent C₁-C₃ radical, preferably a propylene radical;
G₃ represents a polymeric radical resulting from (homo)polymerization of at least one monomer of the carboxylic acid type with ethylene unsaturation, preferably acrylic acid and/or methacrylic acid;
G₄ represents a polymeric radical resulting from (homo) polymerization of at least one monomer of the C₁-C₁₀ alkyl (meth)acrylate type, preferably isobutyl or methyl (meth)acrylate.

Examples of silicone polymers corresponding to the formula (V) are in particular polydimethylsiloxanes (PDMS) on which there are grafted, through a thiopropylene-type secondary linking, mixed polymer moieties of the poly(meth)acrylic acid type and of the alkyl poly(meth)acrylate type.

Other examples of silicone polymers corresponding to formula (IV) are in particular polydimethylsiloxanes (PDMS) on which there are grafted, through a thiopropylene-type secondary linking, polymer moieties of the isobutyl poly(meth)acrylate type.

Such polymers include polymers comprising at least one group of the formula: 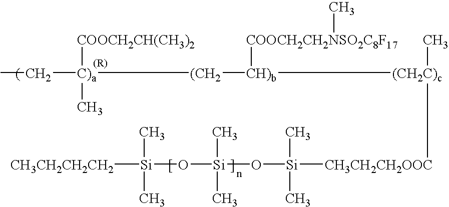
Wherein a, b, and c, which may be identical or different, are each a number ranging from 1 to 100,000; and the terminal groups, which may be identical or different, are each chosen from C₁-C₂₀ linear alkyl groups, C₃-C₂₀ branched chain alkyl groups, C₃-C₂₀ aryl groups, C₁-C₂₀ linear alkoxy groups, and C₃-C₂₀ branched alkoxy groups.

Such polymers are disclosed in U.S. Pat. Nos. 4,972,037, 5,061,481, 5,209,924, 5,849,275, and 6,033,650, and in International patent publications WO 93/23446 and WO 95/06078.

Another family of silicone polymers with a polysiloxane backbone grafted with non-silicone organic monomers particularly well suited to implementation of this invention consists of the silicone polymers comprising in their structure the moiety of formula (VI) below 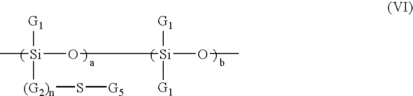
in which the radicals G₁ and G₂ have the same meaning as before; G₅ represents a polymeric residue resulting from (homo)polymerization of at least one monomer of at least one hydrophobic monomer with ethylene unsaturation or copolymerization of at least one anionic monomer with ethylene unsaturation and at least one hydrophobic monomer with ethylene unsaturation; n is equal to 0 or 1; a is a whole number ranging from 0 to 50; and b is a whole number which may range between 10 and 350, on condition that a is other than 0.

The moiety of formula (VI) hereinabove preferably has at least one, and more preferably still all, of the following characteristics:
the radicals G₁ denote an alkyl radical, preferably the methyl radical;
n is not zero, and the radicals G₂ represent a C₁-C₃ divalent radical, preferably a propylene radical.

The molecular mass by number of the silicone polymers with a polysiloxane backbone grafted with non-silicone organic monomers of the invention preferably varies from approximately 10,000 to 1,000,000, and more preferably still from approximately 10,000 to 100,000.

The composition may contain from about 0.1 to about 60% by weight, from about 5 to about 60%, from about 2 to about 30%, or from about 0.1 to about 20% by weight of film-forming polymer dry matter. More generally, the total quantity of polymer should be in sufficient quantity to form on the skin (e.g., eyelids, lips or eyelashes) a cohesive film that is able to follow the movements of the skin and/or the lips without peeling away or cracking.

The composition also may contain at least one hydrophilic or hydrophobic plasticizing agent (or plasticizer), chosen for its compatibility with the polymer or polymers and in a quantity such that it does not impair the sensitivity of the film to water. This agent may be water soluble or insoluble in water and possibly may exist in the form of an aqueous dispersion.

There may be cited in particular, alone or as a mixture, the usual plasticizers such as:
glycols and derivatives thereof such as diethylene glycol ethylether, diethylene glycol methylether, diethylene glycol butylether or even diethylene glycol hexylether, ethylene glycol ethylether, ethylene glycol butylether, ethylene glycol hexylether,
esters of glycerol,
the derivatives of propylene glycol and in particular propylene glycol phenylether, propylene glycol diacetate, dipropylene glycol butylether, tripropylene glycol butylether, propylene glycol methylether, dipropylene glycol ethylether, tripropylene glycol methylether and diethylene glycol methylether, propylene glycol butylether,
esters of acids, in particular carboxylic, such as citrates, phthalates, adipates, carbonates, tartrates, phosphates, sebaceates, and
oxyethylene derivatives such as oxyethylene oils, in particular vegetable oils such as castor oil; and silicone oils.

The quantity of plasticizing agent may be chosen by the person skilled in the art on the basis of his general knowledge, in such manner as to obtain a film having the desired mechanical properties, while preserving the composition of the cosmetically acceptable properties.

According to the invention, the system comprising the film-forming polymer or polymers, the possible coalescent agents and the possible plasticizers shall be called “polymeric system”; this polymeric system is to be capable of forming a film on the support on which it is deposited, supple, flexible, cohesive, following the movements of the support (keratinous tissue, e.g., eyelashes, lips or skin) on which it is deposited.

The compositions of the present invention may further comprise at least one suitable (e.g., cosmetically or dermatologically acceptable) additive or adjuvant, including, for example, colorants, fillers, fibers, sunscreen agents, preservatives, chelators such as EDTA and salts thereof, antioxidants (e.g., BHT, tocopherol), essential oils, fragrances, neutralizing agents, and cosmetically active agents and dermatological active agents such as, for example, anti-inflammatory agents, defoaming agents, emollients, vitamins, trace elements and essential fatty acids. These ingredients may be soluble or dispersible in the aqueous or the fatty phase.

The at least one colorant according to the present invention may be chosen from the lipophilic dyes, hydrophilic dyes, traditional pigments, and nacres usually used in cosmetic or dermatological compositions, and mixtures thereof. The coloring agent may have any shape, such as, for example, spheroidal, oval, platelet, irregular, and mixtures thereof. Pigments may optionally be surface-treated e.g., with silicones, perfluorinated compounds, lecithin, and amino acids.

The liposoluble dyes include, for example, Sudan Red, D&C Red 17, D&C Green 6, β-carotene, soybean oil, Sudan Brown, D&C Yellow 11, D&C Violet 2, D&C Orange 5, quinoline yellow and annatto. The water-soluble dyes are, for example, beetroot juice or methylene blue.

The pigments may be chosen from white pigments, colored pigments, inorganic pigments, organic pigments, coated pigments, uncoated pigments, pigments having a micron size and pigments not having a micron size. Among the inorganic pigments that may be mentioned are titanium dioxide, optionally surface-treated, zirconium oxide, zinc oxide, cerium oxide, chromium oxide, manganese violet, ultramarine blue, chromium hydrate, and ferric blue. Among the organic pigments which may be mentioned are carbon black, pigments of D&C type, lakes based on cochineal carmine, lakes based on barium, lakes based on strontium, lakes based on calcium, and lakes based on aluminium.

The nacreous pigments may, for example, be chosen from white nacreous pigments such as mica coated with titanium and mica coated with bismuth oxychloride, colored nacreous pigments such as titanium mica with iron oxides, titanium mica with, for example, ferric blue and/or chromium oxide, titanium mica with an organic pigment of the type mentioned above, as well as nacreous pigments based on bismuth oxychloride, interferential pigments, and goniochromatic pigments.

Colorants can generally be present in an amount ranging from about 0.01% to about 50% relative to the total weight of the composition.

The compositions of the present invention may also contain dispersion enhancing agents such as polysaccharide resins, e.g., KM 13, available from KAMA International Corp. (Duluth, Ga.). Dispersion enhancing agents are especially preferred in pigmented products.

Fillers and mothers-of-pearl may also be added to the formulations to modify the texture of the composition and the matteness/gloss effect. Fillers should be understood to mean lamellar or non-lamellar, inorganic or synthetic, colorless or white particles. Mothers-of-pearl should be understood to mean irridescent particles produced especially by certain mollusks in their shell or else synthesized. Pearling agents that may be used in the practice of the invention include mica, iron oxides, titanium dioxide and any other pearling agent known in the cosmetic arts.

For example, the fillers and powders may be chosen from those that are well known to a person skilled in the art and that are commonly used in cosmetic compositions. Non-limiting mention may be made of talc, mica, silica, kaolin, polyamide powders, for instance Nylon® (Orgasol from Atochem), poly-β-alanine powders and polyethylene powders, tetrafluoroethylene polymer powders, for instance Teflon®, lauroyl lysine; starch, boron nitride, hollow polymer microspheres such as those of polyvinylidene chloride/acrylonitrile, for instance Expancel® (Nobel Industrie), acrylic powders such as Polytrap® (Dow Corning), polymethyl methacrylate particles and silicone resin microbeads (for example Tospearls® from Toshiba), magnesium hydrocarbonate, hydroxyapatite, hollow silica microspheres (Silica Beads® from Maprecos), glass or ceramic microcapsules, metal soaps derived from carboxylic organic acids comprising from 8 to 22 carbon atoms, such as from 12 to 18 carbon atoms, for example zinc stearate, magnesium stearate, lithium stearate, zinc laurate and magnesium myristate, and mixtures thereof, chalk, Fuller's earth, sericite, muscovite, phlogopite, lepidolite, biotite, vermiculite, magnesium carbonate, calcium carbonate, aluminum silicate, smectite clays, alkyl and/or trialkyl aryl ammonium smectites, chemically modified magnesium aluminum silicate, organically modified montmorillonite clay, hydrated aluminum silicate, fumed silica, aluminum starch octenyl succinate, barium silicate, calcium silicate, magnesium silicate, strontium silicate, metal tungstate, magnesium, alumina, zeolite, barium sulfate, calcined calcium sulfate (calcined gypsum), calcium phosphate, colloidal silicone dioxide, boron nitride, cyclodextrin, benzoguanamine resin powder, and cellulose powders such as hydroxyethyl cellulose and sodium carboxymethyl cellulose.

Filler(s) may be present in an amount ranging from about 0.1% to about 25%, for example, from about 1% to about 20% by weight of the total weight of the composition.

In some embodiments, such as in mascara, the composition further comprises fibers to allow an improvement in the lengthening effect. The fibers useful in the present invention may be chosen from natural and synthetic fibers. Natural fibers include, but are not limited to, cotton, silk, wool, and other keratin fibers. Synthetic fibers include, but are not limited to, polyester, rayon, nylon and other polyamide fibers.

The fibers may be present in the compositions in an amount generally ranging from about 0.01% to about 10% by weight of the composition.

Representative examples of preservatives include alkyl para-hydroxybenzoates, wherein the alkyl radical has from 1, 2, 3, 4, 5 or 6 carbon atoms and preferably from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben), and phenoxyethanol. Mixtures of preservatives may certainly be used, e.g., the mixture of methyl-paraben, ethylparaben, propylparaben and butylparaben sold under the name Nipastat by Nipa, and the mixture of phenoxyethanol, methylparaben, ethylparaben, propylparaben and butylparaben sold under the name Phenonip, also by Nipa. These preservatives may be present in amounts generally ranging from about 0.01% to about 15% by weight of the composition.

The following examples are intended to further illustrate the present invention. They are not intended to limit the invention in any way. Unless otherwise indicated, all parts are by weight.

### EXAMPLE 1

### Eyeshadow

| Seq | | |
|---|---|---|
| A | Butylene Glycol | 7.00 |
| | Methylparaben | 0.35 |
| B | Water | 69.60 |
| | Glycerin | 5.00 |
| C | Xanthan Gum and | 0.75 |
| | Locust Bean Gum | |
| | (1:1) | |
| D | PVP K90 | 1.50 |
| E | Water | 3.00 |
| | Panthenol | 0.50 |
| F | Pigments/fillers | 11.80 |
| G | Phenoxyethanol | 0.5 |
| | | 100.00 |

To make the eyeshadow, phase A ingredients, butylene glycol and methylparaben, were dispersed in the main beaker using a stir bar and heated to 50-55° C. Once dispersed phase B ingredients, water and glycerin, were added and mixed with a paddle mixer until dispersed. The Xanthan and the locust bean gum, i.e., phase C ingredients, were sprinkled in, and mixed for about 90 minutes at 80-85° C. Phase D ingredient, film-forming polymer PVP K90D was added at 80° C. When the gel was at a temperature of 80° C., phase F ingredients were added using a paddle mixer, and were mixed slowly until homogeneous. At 80° C., phase E ingredients, water and panthenol, followed by addition of phase G, i.e., phenoxyethanol, were dispersed into the mixture. The resultant liquid eyeshadow composition was filled into a pot at about 60-70° C. The rest of the batch was dropped.

### EXAMPLE 2

### Mascara

| SEQ | INCI Name | |
|---|---|---|
| A1 | Waxes | 13.20 |
| | Glyceryl Stearate | 4.000 |
| | Stearic acid | 5.000 |
| | Butylparaben | 0.050 |
| | PVP/Eicosene | 3.000 |
| | copolymer | |
| | ETHYLENEDIAMINE/TALL | 6.270 |
| | OIL DIMER | |
| | ACID/STEARYL ALCOHOL | |
| | COPOLYMER | |
| A2 | Pigments and fillers | 7.000 |
| B | Water | 47.780 |
| | Xanthan Gum and | 0.100 |
| | Locust Bean Gum (1:1) | |
| | PVP K-30 | 1.000 |
| | Butylene Glycol | 2.000 |
| | Methylparaben | 0.400 |
| | Triethanolamine | 2.000 |
| | Simethicone | 0.100 |
| C | Cyclopentasiloxane | 1.50 |
| | Cyclopentasiloxane/ | 2.00 |
| | Dimethiconol | |
| D | D.I. Water | 1.010 |
| | POLYQUATERNIUM-10 | 0.100 |
| | Bisabolol | 0.190 |
| E | Acrylates copolymer | 2.300 |
| | Film former | |
| F | Panthenol | 0.500 |
| G | Phenoxy Ethanol | 0.500 |
| | TOTAL | 100.000 |

To make the mascara, phase A1 ingredients were mixed together and heated to 95° C.-100° C. Upon achieving a uniform dispersion, pigments and fillers (A2) were added to A1 at 90-95° C. To make phase B, xanthan gum and locust bean gum were added to water at room temperature, and mixed with a lighting mixer for 30 minutes (or until dissolved), followed by addition of PVP K30, while mixing at room temperature until dispersed. These ingredients were heated to 80° C., followed by addition of pre-mixed butylene glycol and methylparaben (which were mixed at about 70° C.), followed by consecutive addition of triethanolamine and simethicone until dispersion was achieved. Phase B was continued to be heated to 85-90° C. Phase B was added to the mixture of phases A1 and A2 at 90-95° C., and emulsified for 20 minutes under a homogenizer, followed by switching to a paddle mixer and air cooling slowly to 60° C. At that time, phase C ingredients were slowly added. At 55° C., pre-mixed phase D ingredients (water and Polyquaternium 10, which was cooled to below 45° C. prior to addition) were added slowly, followed by slow addition of Bisabolol. At 50° C., the acrylates co-polymer was slowly added, followed by slow addition, at 45° C. of phase F ingredient (panthenol), and then followed at 40° C., by slow addition of phase G, and then dropping the batch to a temperature of 32-35° C.

All publications cited in the specification are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A cosmetic composition comprising an aqueous phase, a liquid fatty phase, a structuring agent, a film-forming polymer, xanthan gum and a non-gelling polysaccharide.

2. The composition of claim 1, wherein said non-gelling polysaccharide comprises locust bean gum.

3. The composition of claim 2, wherein xanthan gum and locust bean gum are present in a ratio of about 1:1 based on dry weight.

4. The composition of claim 1, wherein the non-gelling polysaccharide comprises konjac mannan.

5. The composition of claim 1, wherein the non-gelling polysaccharide comprises guar gum.

6. The composition of claim 1, wherein the structuring agent comprises at least one wax.

7. The composition of claim 6, wherein said wax is present in an amount of at least 10% by weight of the composition.

8. The composition of claim 6, comprising at least two waxes.

9. The composition of claim 1, wherein said oil comprises a volatile oil.

10. The composition of claim 9, wherein said volatile oil comprises isododecane.

11. The composition of claim 1, wherein said oil comprises petroleum distillate.

12. The composition of claim 1, further comprising a colorant.

13. The composition of claim 1, further comprising a filler.

14. The composition of claim 1, further comprising a moisturizing agent.

15. The composition of claim 14, wherein said moisturizing agent comprises glycerin.

16. The composition of claim 1, further comprising fibers.

17. The composition of claim 1, wherein said film-forming polymer comprises an acrylates copolymer or PVP or a copolymer of PVP.

18. The composition of claim 1, wherein said film-forming polymer is present in an amount of about 0.1 to about 20% by weight of the composition.

19. The composition of claim 1, which is a mascara composition.

20. A method of extending wear and/or enhancing volume of eye lashes, comprising applying to eyelashes a mascara composition comprising xanthan gum and a non-gelling polysaccharide.

21. A method for applying make-up to eyelids or lips comprising applying to eye lids or lips a cosmetic composition comprising xanthan gum and a non-gelling polysaccharide.
